(19) United States
(12) Plant Patent Application Publication
(10) Pub. No.: US 2006/0162034 P1
(43) Pub. Date: Jul. 20, 2006

(54) GYPSOPHILA PLANT NAMED 'DANGYPWHIFA'

(50) GYPSOPHILA PANICULATA; GYPSOPHILA

(75) Inventor: Gabriel Danziger, Moshav Nir-Zvi (IL)

Correspondence Address: FOLEY AND LARDNER LLP;SUITE 500, 3000 K STREET NW, WASHINGTON, DC, 20007, US

(73) Assignee: Danziger "Dan" Flower Farm

(21) Appl. No.: 11/036,337

(22) Filed: Jan. 18, 2005

(51) Int. Cl. A01H 5/00

(52) U.S. Cl. PLT/354

(57) ABSTRACT

A new and distinct Gypsophila plant named ‘Dangypwhifa’ characterized by having globular-shaped, double flowers, 10-11 mm in diameter; conic, bright white flowers arranged in clusters, creating a wide and impressive conic structure; 7-9 stems per plant in first flash; medium flash duration; erect, stable growth habit, branching from lower part of main stem, 80-90 cm in height; uniformly good quality of inflorescence year round; good post-production longevity, with vase life of 10-14 days; and commerically suitable product, marketed as a cut flower.

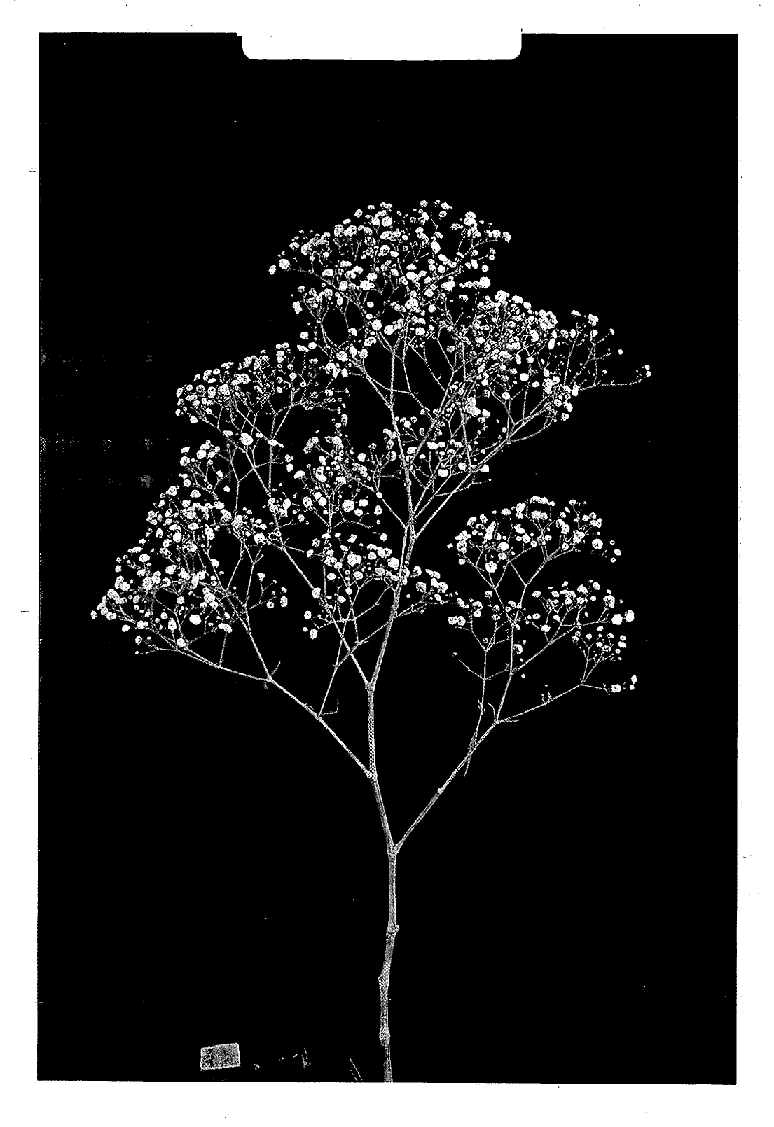

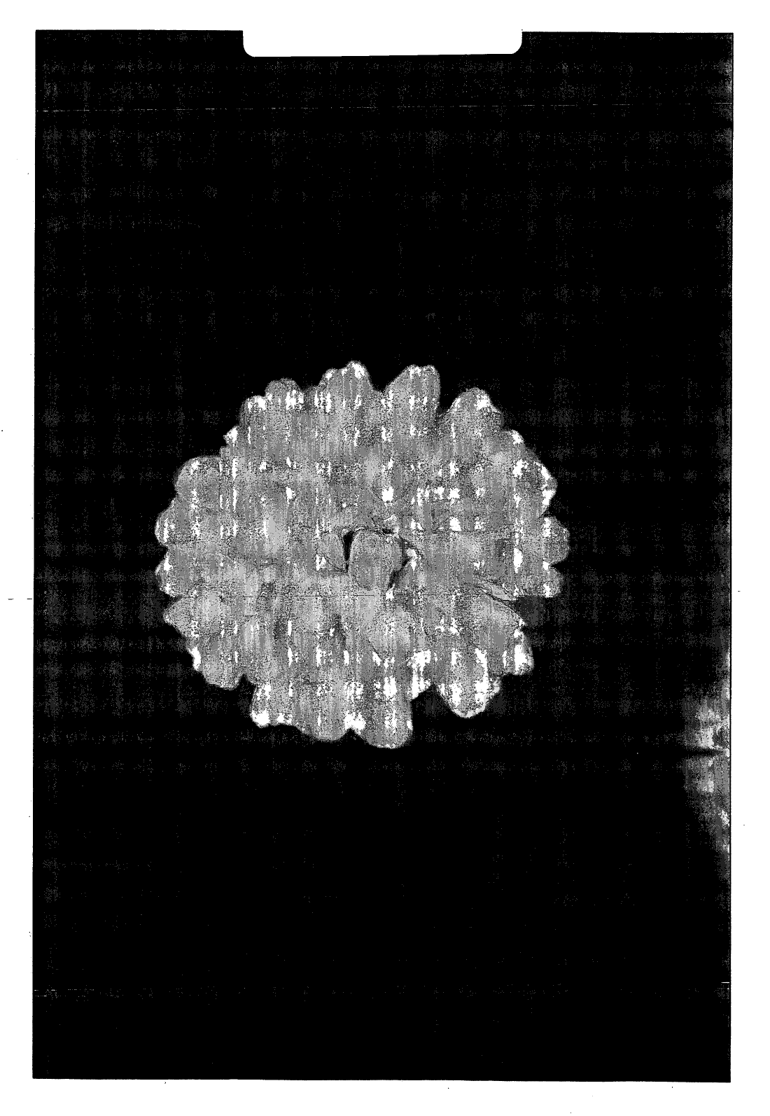

### LATIN NAME OF THE GENUS AND SPECIES OF THE CLAIMED PLANT

Gysophila paniculata (hybrid)

### VARIETY DENOMINATION

‘Dangypwhifa’

### BACKGROUND OF THE INVENTION

The present invention relates to a new and distinct cultivar of Gypsophila plant, botanically known as Gypsophila paniculata, hereinafter referred to by the cultivar name ‘Dangypwhifa’.

Gypsophila, of the Carophyllaceae family, consists of hardy annuals and perennials which are grown in various parts of the United States and Europe. Gypsophila plants are commonly called eithers Baby's Breath of Chalk Plant.

The new Gypsophila cultivar is a product of a planned breeding program conducted by the inventor, Gabriel Danziger, in Moshav Mishmar Hashiva, Israel. The objective of the breeding program is to develop a new Gypsophila variety with large, globular-shaped, double, bright white flowers; good vase life; and ease in harvesting variety.

The new Gypsophila cultivar was originated from a cross made in a controlled breeding program by the inventor in May of 2001, in Moshav Mishmar Hashiva, Israel. The female parent is the hybrid line designated ‘CV.2088’ (unpatented). The male parent is the hybrid line designated ‘CV-184’ (unpatented). The new Gysophila cultivar ‘Dangypwhifa’ was discovered and selected by the inventor, Gabriel Danziger, as a flowering plant within the progeny of the stated cross in a controlled environment in Moshav Mishmar Hashiva, Israel.

Asexual reproduction of the new cultivar by removing cutting from the initial plant was first performed in March of 2002 in Moshav Mishmar Hashiva, Israel, and has demonstrated that the combination of characteristics as herein disclosed for the new cultivar are firmly fixed and retained through successive generations of asexual reproduction.

### BRIEF DESCRIPTION OF THE INVENTION

The following traits have been repeatedly observed and are determined to be unique characteristics of ‘Dangypwhifa’ which in combination distinguish this Gypsophila as a new and distinct cultivar:

1) Globular-shaped, double flower, 10-11 mm in diameter;

2) Conic, bright white flowers arranged in clusters, creating a wide and impressive conic structure;

3) 7-9 stems per plant in first flash;

4) Medium flash duration;

5) Erect, stable growth habit, branching from lower part of main stem, 80-90 cm in height;

6) Uniformly good quality of inflorescence year round;

7) Good post-production longevity, with vase life of 10-14 days; and

8) Commerically suitable product, marketed as a cut flower.

Table 1 provides a comparison among plants of ‘Dangypwhifa’ and plants of the parents, ‘CV-184’ and ‘CV.2088’:

TABLE 1

| | | Female Parent | Male Parent |
|---|---|---|---|
| Trait | ‘Dangypwhifa’ | ‘CV.2088’ | ‘CV-184’ |
| Flower | Width: 9-12 mm | Width: 9-12 mm | Width: |
| | | | 9-12 mm |
| | Color: | Color: white | Color: white |
| | bright white | | |
| | Vaselife: 10-12 | Vaselife: 10-12 days | Vaselife: |
| | days | | 10-12 days |
| Inflorescence | Conic shape | Conic shape | Conic - |
| | | | open shape |
| | 900-1000 flowers | 900-1000 flowers per | 900-1000 |
| | per flowering | flowering stem | flowers per |
| | stem | | flowering stem |
| Yield | 7-9 | 6-7 | 8-9 |
| (stems per | | | |
| plant in | | | |
| first | | | |
| flash) | | | |
| Growth | Bush, branching | Bush, branching from | Bush |
| Habit | from plant base | plant base | Height: |
| | Height: 80-90 cm | Height: 80-90 cm | 70-80 cm |
| Flowering | Medium | Medium | Medium |
| time | | | |

Of the many commercial cultivars known to the present inventor, the most similar in comparison to ‘Dangypwhifa’ is the Gysophila cultivar ‘Danapurna’ (unpatented). In side-by-side comparisons conducted in Moshav Mishmar Hashiva, Israel, plants of ‘Dangywhifa’ differed from plants of ‘Danapurna’ in the following characteristics provided in Table 2.

TABLE 2

| Characteristic | ‘Dangypwhifa’ | ‘Danapurna’ |
|---|---|---|
| Flower color | White group, RHS | White group, RHS |
| | 155D | 155D |
| Inflorescence | Conic | Flowers concentrate |
| Formation | | at upper part |
| Yield (stems | 7-9 | 10-12 |
| per plant in | | |
| first flash) | | |
| Stem height | 80-90 | 70-80 |
| (cm) | | |
| Flowering | 1 week less than | Medium |
| time | ‘Danapurna’ | |

### BRIEF DESCRIPTION OF THE DRAWING

The accompanying photographic drawings illustrate the overall appearance of the new Gypsophila cultivar ‘Dangypwhifa’ showing the colors as true as is reasonably possible with colored reproductions of this type. Colors in the photographs may differ slightly from the color value cited in the detailed botanical description which accurately describe the color of ‘Dangypwhifa’.

The first photograph shows a side view perspective of a flowering stem of ‘Dangypwhifa’. The second photograph shows a close-up view of a single flower of ‘Dangypwhifa’.

### DETAILED BOTANICAL DESCRIPTION

The new Gypsophila cultivar ‘Dangypwhifa’ has not been observed under all possible environmental conditions. The phenotype of the new cultivar may vary significantly with variations in environment such as temperature, light intensity, and day length without any change in the genotype of the plant.

The aforementioned photographs, together with the following observations, measurements and values describe the new Gypsophila cultivar as grown in a greenhouse in Moshav Mishmar Hashiva, Israel, under conditions which closely approximate those generally used in commercial practice.

Color references are made to The Royal Horticultural Society Colour Chart (RHS), except where general colors of ordinary significance are used. Color values were taken under daylight conditions at approximately 12:00 p.m. in Moshav Mishmar Hashiva, Israel. The age of the plant described is 10 weeks.
Classification:
   Botanical.—Hybrid of Gypsophila paniculata.
   Commercial.—Gypsophila ‘White Fire’.
Parentage:
   Female parent.—Hybrid line designated ‘CV.2088’ (unpatented).
   Male parent.—Hybrid line designated ‘CV-184’ (unpatented).
Origin: F1 hybrid.
Propagation: Stem cutting.
Plant:
   General appearance and form.—Height: 80-90 cm. Width: 50-60 cm. Habit: Weak, apical dominance, branching from plant base. Form: Upright bush type.
   Flowering response.—Long day.
   Flowering season.—Naturaly during summer. All year with supplemental lighting.
   Winter hardiness.—Half-hardy.
   Lastingness of the individual bloom.—10-12 days.
   Rooting.—Use rooting hormone.
   Fragrance.—Typical to Gypsophila.
Foliage:
   Overall shape of leaf.—Lanceolate.
   Base.—Truncate.
   Tip.—Acuminate.
   Margin.—Entire.
   Texture.—Rough.
   Main color of upper surface.—Mature leaf: Green, RHS 137 A. Immature leaf: Green, RHS 137 A.
   Main color of lower surface.—Mature leaf: Green, RHS 137 B. Immature leaf: Green, RHS 137 B.
   Venation color.—Upper surface: None. Lower surface: None.
   Size.—Length: 9-10 cm. Width: 1.3-1.5 cm.
   Stipules.—None.
   Petiole.—None.
Inflorescence:
   Natural flowering season.—Long day.
   Corolla.—Form: Double flower. Shape: Globular. Average Number: 900-1000 flowers per flowering stem. Size: 10-11 mm in diameter.
   Petal.—Petal Number: 45-50. Petal Shape: Spatulate with tip emarginated. Petal Markings: None. Length: 4-6 mm. Width: 1-2 mm. Petal Color: Upper surface: White, RHS 155 D. Lower surface: White, RHS 155 D.
   Sepal.—Sepal Shape: Oval with pointed tip. Length: 3-4 mm. Width: 1.5 mm. Margin: Both surfaces: Transparent, color: white group, RHS 155 D. Sepal Color: Upper surface: Green group, RHS 137 C. Lower surface: Green group, RHS 137 B.
   Stem.—Average length: 80-90 cm. Average diameter: 5-6 mm. Internode length: 4-6 cm. Color: Yellow-Green, RHS 144 B.
   Spur.—None.
   Bud.—Response: Long day. Color: Yellow group, RHS 2 D. Size before opening: Height: 3 mm. Diameter: 4 mm. Shape: Oblate.
   Pedicel.—Pedicel Length: 8-10 mm. Pedicel Color: Yellow-Green, RHS 146 A.
Reproductive organs:
   Androecium.—
   Stamen.—Number: 5-10 (seldom seen). Color: White group, RHS 155D (transparent).
   Anthers.—Number: 5-10 (seldom seen). Color: Orange-white group, RHS 159 D.
   Pollen.—Amount: A lot. Color: Orange-white group, RHS 159 D.
   Gynoecium.—
   Pistil.—Number: 2. Shape: Convex. Color: White group, RHS 155 D (transparent).
   Ovary.—Color: Yellow-green group, RHS 144 D.
   Seeds.—Width: 1 mm. Length: 1 mm. Shape: Kidney-shaped. Texture: Canescent. Color: Brown group, RHS 200 A.
   Fruit.—Width: 2-3 mm. Length: 2-3 mm. Shape: Oblate with conical tip. Color: Greyed-yellow group, RHS 161 A.
Disease resistance: No information is currently available.
Disease susceptibility: No information is currently available.

I claim:

1. A new and distinct Gypsophila plant named ‘Dangypwhifa’, substantially as illustrated and described herein.
